(19) United States
(12) Patent Application Publication
(10) Pub. No.: US 2025/0243133 A1
(43) Pub. Date: Jul. 31, 2025

(54) IMPROVED PROCESS FOR THE PRODUCTION OF MIDDLE DISTILLATES BY OLIGOMERIZATION OF AN OLEFINIC FEEDSTOCK

(71) Applicant: AXENS, Rueil-Malmaison (FR)

(72) Inventor: Pierre-Antoine GEORG, Rueil-Malmaison (FR)

(73) Assignee: AXENS, Rueil-Malmaison (FR)

(21) Appl. No.: 18/854,277

(22) PCT Filed: Apr. 4, 2023
(86) PCT No.: PCT/EP2023/058741

§ 371 Date: Oct. 4, 2024

(30) Foreign Application Priority Data
Apr. 5, 2022 (FR) FR2203112

(51) Int. Cl. C07C 2/12; C07C 7/09

(52) U.S. Cl. CPC C07C 2/12; C07C 7/09; C07C 2529/06

(57) ABSTRACT

A process for preparing middle distillates from an olefinic feedstock, by
a) oligomerization fed with the olefinic feedstock, a first recycle and a second recycle, and operated in the presence of at least one oligomerization catalyst, to produce a reaction effluent containing dimers, trimers and oligomers;
b) fractionating the reaction effluent into:
   a light fraction containing at least part of the unconverted olefinic feedstock;
   an intermediate fraction containing at least part of the dimers and trimers; and
   a heavy fraction containing the oligomers;
c) recycling by the preparation of a first recycle containing at least part of the light fraction; and a second recycle containing at least part of the intermediate fraction; and transfer of the first recycle and the second recycle to step a);
d) hydrogenating at least part of the heavy fraction.

### TECHNICAL FIELD

The present invention relates to a process for the production of middle distillates, in particular kerosene and/or gas oil, meeting the specifications in force, in particular those defined in the standard ASTM D1655 or ASTM D7566 for kerosene and those defined in the standard ASTM D975 or the European standard 15940 for gas oil, by heterogeneous oligomerization of an olefinic feedstock, in particular a biobased olefinic feedstock.

### PRIOR ART

Airlines companies committed to carbon-neutral growth particularly in commercial aviation from 2021, and American airlines companies set an objective to reduce CO₂ emissions by 50% in 2050 relative to the 2005 levels. However, the improvements in aircraft and engine efficiency are not proving to be sufficient to attain carbon neutrality. Sustainable aviation fuels (or SAF) therefore appear to be critical for achieving this objective.

It therefore appears necessary to develop methods for manufacturing synthetic kerosene from preferentially biobased feedstocks.

Patent FR 2926812 thus discloses an olefin oligomerization process, for producing fuel, for example for producing gasoline and/or kerosene from light olefinic feedstocks containing between 2 and 8 carbon atoms (C2-C8), and in particular from light olefinic feedstocks containing high proportions of propylene and/or butenes and/or pentenes, and using an oligomerization catalyst based on, and preferably consisting solely of, silica-alumina with a reduced macropore content.

Patent EP 1 396 532, for its part, describes a process for upgrading a liquid hydrocarbon feedstock, involving: a) separating from said hydrocarbon feedstock a fraction (O1) essentially comprising compounds containing 5 carbon atoms (C5), of which at least 2% by weight are pentenes; b) placing said fraction (O1) in contact with a hydrocarbon cut (02) comprising hydrocarbons having a number of carbon atoms between 6 and 10 (C6-C10), of which at least 2% by weight are olefins, in the presence of an acid catalyst promoting the dimerization and alkylation reactions of the species; c) separating the effluent obtained into at least two cuts, including a gasoline cut (a) with a top distillation point below 100° C. and comprising the majority of the unreacted reagents, and a kerosene cut (B) with a distillation range between 100° C. and 300° C.

Patent EP 1 602 637 describes a process for simply and economically modulating the respective productions of gasoline and gas oil fuel, by transforming the initial feedstock of hydrocarbons comprising 4 to 15 carbon atoms (C4-C15) into a gasoline fraction with an improved octane number compared to that of the feedstock and a gas oil fraction with a high cetane number.

Patent EP 1 739 069 describes a process for preparing a gas oil cut from an olefinic feedstock containing 2-12 carbon atoms (C2-C12), comprising two oligomerization steps with a separation step in between. The intermediate separation step produces a light cut of C4-C5 olefinic hydrocarbons, an intermediate cut with a T95 of between 180° C. and 240° C. and a heavy cut with a T95 greater than 240° C. The intermediate cut is then mixed with at least a fraction of the light cut in a mass ratio (intermediate cut/light cut) of between 60/40 and 80/20 and then undergoes a second oligomerization.

Patent EP 2 385 092 describes a process for producing middle distillate hydrocarbon bases from ethanol, more particularly from bioethanol.

Patent EP 2 707 462 discloses a process for the oligomerization of olefins comprising from 4 to 6 carbon atoms (C4-C6) into a middle distillate cut predominantly containing from 10 to 20 carbon atoms (C10-C20). In the process of patent EP 2 707 462, the starting olefinic feedstock must comprise a minimum of branched olefins (or iso-olefins), preferably at least 10% by weight and preferably at 20% by weight of iso-olefins relative to the total olefins in the feedstock.

Patent FR 2 959 750 describes a process for producing middle distillate hydrocarbon bases, preferably kerosene hydrocarbon base, from an ethanol feedstock derived from biomass, said process comprising the dehydration of ethanol into a predominantly ethylenic effluent, two successive oligomerization steps to obtain a middle distillate effluent.

Patent FR 3 053 355 describes a process for the oligomerization of light olefinic feedstocks containing between 2 and 10 carbon atoms (C2-C10) per molecule, using a catalytic system comprising a silica-alumina catalyst and a zeolite catalyst having pore apertures of 10 or 12 oxygen atoms, and performed at a temperature of between 13° and 350° C., at a pressure of between 0.1 and 10 MPa and at an HSV (hourly space velocity) of between 0.1 and 5 h 1. The process of EP 3 053 355 improves the yield of middle distillates and in particular the yield of gas oil, compared to an oligomerization process using only one of the catalysts of the catalytic system used, for an equivalent volume of catalyst.

Patent U.S. Pat. No. 6,372,949 describes the conversion of oxygen-based compounds into gasoline and distillates (C4-C12 cut) in a single dehydration-oligomerization step using a composite catalyst comprising a one-dimensional 10 MR zeolite chosen from the group consisting of ZSM 22, ZSM 23, ZSM 35, ZSM 48, ZSM 57 and ferrierite and mixtures thereof, with a multidimensional zeolite having a medium pore size, such as the zeolite ZSM-5.

However, none of the processes of the prior art discloses a means for producing middle distillates, in particular kerosene and/or gas oil, from C3 to C6, preferably C3 to C4, light olefins, notably obtained from biobased feedstocks, in a very specific manner and with improved yields of middle distillates, in particular kerosene and/or gas oil, which meet the specifications in force, in particular the specifications of the standard ASTM D7566 and/or of the European standard 15940, while at the same time maintaining a very satisfactory or even high conversion of the olefinic feedstock.

### SUMMARY OF THE INVENTION

Thus, the present invention relates to a process for preparing middle distillates from an olefinic feedstock from an olefinic feedstock which comprises monoolefins containing between 3 and 6 carbon atoms, wherein the process comprises:
a) an oligomerization step which is fed with at least the olefinic feedstock, a first recycle and a second recycle, and which is operated in the presence of at least one oligomerization catalyst, at a temperature of between 2° and 500° C., a pressure of between 1.0 and 10 MPa and an HSV between 0.1 and 0.5 h 1, to produce a reaction effluent comprising dimers, trimers and oligomers;
b) a step of fractionating the reaction effluent obtained at the end of step a), into at least:
   a light fraction comprising at least part of the olefinic feedstock not converted in step a);
   an intermediate fraction comprising at least part of the dimers and trimers produced in step a); and
   a heavy fraction comprising the oligomers;
c) a recycle step, comprising: preparing a first recycle which comprises at least part of the light fraction; preparing a second recycle which comprises at least part of the intermediate fraction; and transferring the first recycle and the second recycle to the oligomerization step a);
d) a step for hydrogenating at least part of the heavy fraction separated out in step b) in the presence of hydrogen, to give a hydrogenated heavy fraction comprising middle distillates.

The value of the process according to the invention is that it proposes a process for an efficient conversion of light olefinic feedstocks, in particular comprising olefins containing between 3 and 6, preferably between 3 and 4, carbon atoms, and which are more particularly at least partly biobased, in order to selectively produce a middle distillate cut, and more particularly a kerosene cut or a gas oil cut, which meet the specifications in force and notably the specifications of the standard ASTM D7566 or the European standard 15940, respectively.

The process according to the invention also makes it possible to greatly improve the selectivity towards middle distillates, or even more particularly towards kerosene or gas oil, compared with the oligomerization processes of the prior art, and thus to maximize the yields of middle distillates, more particularly of kerosene or gas oil, while at the same time maintaining a satisfactory, or even high, overall conversion of the starting olefinic feedstock.

Another advantage of the process according to the invention lies in the fact that any type of feedstock and in particular biobased olefinic feedstocks which typically comprise a high proportion of olefins and are thus highly reactive, can be converted into hydrocarbon products and in particular with high yields of middle distillates, and more particularly of kerosene or gas oil.

### DESCRIPTION OF THE EMBODIMENTS

According to the present invention, the expressions “of between . . . and . . . ” and “between . . . and . . . ” are equivalent and mean that the limit values of the interval are included in the described range of values. If such is not the case and if the limit values are not included in the range described, such information will be introduced by the present invention.

For the purposes of the present invention, the various ranges of parameters for a given step, such as the pressure ranges and the temperature ranges, may be used alone or in combination. For example, within the meaning of the present invention, a range of preferred pressure values can be combined with a range of more preferred temperature values.

In the continuation of the text, specific embodiments of the invention may be described. They can be implemented separately or combined together, without limitation of combinations when this is technically feasible.

The terms “upstream” and “downstream” should be understood as a function of the general flow of the stream(s) under consideration in the process.

The term “biobased” means that the material/product/compound which it qualifies is an organic material/product/compound whose carbon originates from CO₂ present in the atmosphere that has been fixed recently (on the human scale) by means of solar energy (photosynthesis). Terrestrially, this CO₂ is captured or fixed by plant life (for example, agricultural crops or forest materials). In the oceans, the CO₂ is captured or fixed by photosynthesizing bacteria or phytoplankton. For example, a biobased material has a ¹⁴C/¹²C isotope ratio of more than 0. Conversely, a material of fossil origin has a ¹⁴C/¹²C isotope ratio of about 0. The terms “renewable” or “obtained from renewables” may also be used. To determine whether a material/product/compound is biobased or obtained from renewables, its modern carbon content (or percent modern carbon, pMC) is measured according to the standard ASTM D 6866-21 (“Determining the biobased content of natural-range materials via analysis by isotope and radiocarbon ratio mass spectrometry”). The method of this standard indeed measures the ¹⁴C/¹²C isotope ratio in a sample and compares it with the ¹⁴C/¹²C isotope ratio of a standard biobased reference to give the percentage biobased content of the sample, the reference giving a radiocarbon content roughly equivalent to the fraction of atmospheric radiocarbon in 1950. The pMC of the standard biobased reference material therefore equals 100%. The pMC of a biobased material is strictly greater than 0%, for example greater than or equal to 1%. The pMC of a material of fossil origin is around 0%. A current biobased material may therefore also possibly have a pMC of more than 100%.

In the present description, the terms “T95” or “T95 temperature” are interchangeable and denote the temperature at which 95% by weight of the product in question has evaporated. It is determined according to the standardized method ASTM D2887. In parallel, “T5” or “T5 temperature” is the temperature at which 5% by weight of the product in question has evaporated, determined according to the same standardized method ASTM D2887.

In the present description, the term “Cx” denotes compounds including x carbon atoms. For example, a C3 chemical compound contains 3 carbon atoms. The term “Cx+” denotes compounds containing at least x carbon atoms. For example, C9+ compounds are compounds containing at least 9 carbon atoms (i.e. 9 or more carbon atoms). The term “Cx-” denotes compounds containing at most x carbon atoms.

Throughout the present text, the groups of chemical elements are described according to the new IUPAC classification. For example, Groups 9 or 10 correspond to the metals of respectively columns 9 and 10 according to the IUPAC classification or to the last two columns of Group VIIIB according to the CAS classification (CRC Handbook of Chemistry and Physics, CRC editor press, chief editor D.R. Lide, 81st edition, 2000-2001). Similarly, Group 6 corresponds to the metals of column 6 according to the IUPAC classification or to the metals of columns VIB according to the CAS classification.

According to the present invention, the terms “olefin” and “monoolefin” are used without distinction from one another and refer to hydrocarbons comprising a double bond. Preferably, the olefins of the olefinic feedstock of the process comprise between 3 and 6 carbon atoms (C3-C6), preferably 3 and/or 4 carbon atoms (C3 and/or C4). The olefins obtained after oligomerization preferably comprise between 6 and 30 carbon atoms (C6-C30), preferably between 9 and 25 carbon atoms (C9-C25), in particular between 9 and 16 carbon atoms (C9-C16) or between 10 and 25 carbon atoms (C10-C25).

According to the present invention, the term “oligomerization” denotes any addition reaction of one olefin onto another olefin, until compounds are obtained, in particular hydrocarbon compounds, more particularly mono-olefinic hydrocarbon compounds, typically containing between 6 and 30 carbon atoms, preferably between 9 and 25 carbon atoms (C9-C25), in particular between 9 and 16 carbon atoms (C9-C16) or between 10 and 25 carbon atoms (C10-C25). Thus, the products obtained are dimers or trimers of the olefins of the olefinic feedstock, i.e. olefinic compounds resulting from the condensation of two or three olefinic molecules of the olefinic feedstock, respectively, or oligomers which correspond to olefinic compounds resulting from the condensation of several olefinic molecules of the olefinic feedstock (“several” meaning here more than 3 but less than 10, preferably less than or equal to 5, preferably less than or equal to 4). Typically, oligomers are obtained from C4 olefins, the number of carbon atoms of which is largely less than or equal to 30, and most preferably between 9 and 25, in particular between 9 and 16 or between 10 and 25. Oligomerization is distinguished from polymerization by the addition of a limited number of molecules. The number of molecules added in the context of the invention is between 2 and 10, preferentially between 3 and 6, and even more preferably between 3 and 6. The oligomers may, however, comprise traces of olefins which have been oligomerized with a number of molecules greater than 10. Usually, these traces represent less than 5% by weight relative to the oligomers formed.

The term “heterogeneous catalysis” defines, in the present disclosure, a reaction, in particular oligomerization reactions, where at least two phases coexist, the catalyst being in solid form. In particular, the oligomerization step of the process according to the invention involves the oligomerization of the olefinic feedstock by heterogeneous catalysis, i.e. in the presence of a catalyst in solid form, the feedstock and advantageously the products obtained preferably being in liquid phase.

More particularly, the present invention relates to a process for preparing middle distillates, preferably a kerosene cut and/or a gas oil cut, from a C3 to C6 olefinic feedstock, preferably a C3 to C4 and in particular C3 or C4 feedstock or mixtures thereof, comprising, preferably consisting of:
a′) optionally a step of pretreating the olefinic feedstock, preferably using at least one adsorption section, a water washing section, a hydrotreating section and/or a selective hydrogenation section;
a″) optionally a step of separating the olefinic feedstock to separate at least partially the C5 and C6 compounds present in said olefinic feedstock;
a) an oligomerization step which is fed with at least the olefinic feedstock, possibly pretreated and/or separated, a first recycle and a second recycle, and which is operated, preferably in the liquid phase, in the presence of at least one preferably solid oligomerization catalyst at a temperature preferably between 2° and 500° C., at a pressure preferably between 1.0 and 10 MPa, and an HSV preferably between 0.1 and 0.5 h 1, to produce a reaction effluent comprising dimers, trimers and oligomers;
b) a step of fractionating the reaction effluent obtained at the end of step a), into at least:
   a light fraction comprising at least part of the olefinic feedstock not converted in step a);
   an intermediate fraction comprising at least part of the dimers and trimers advantageously produced in step a); and
   a heavy fraction, comprising the oligomers present in the reaction effluent obtained from step a);
c) a recycle step, comprising: the preparation of a first recycle comprising, preferably consisting of, at least part of the light fraction obtained from the fractionation step b); the preparation of a second recycle comprising, preferably consisting of, at least part of the intermediate fraction obtained from the fractionation step b); and the transfer of the first recycle and the second recycle to the oligomerization step a);
d) a step of hydrogenating at least part of the heavy fraction separated out in step b) in the presence of hydrogen, to give a hydrogenated heavy fraction advantageously comprising at least one middle distillate cut;
e) optionally a step of separating out the hydrogenated heavy fraction, to obtain at least one middle distillate cut, in particular a kerosene cut and/or a gas oil cut.

### The Olefinic Feedstock

The feedstock treated by means of the process according to the invention is advantageously a “light olefinic feedstock”, i.e. comprising hydrocarbon compounds and in particular olefins, preferably mono-olefins, containing between 3 and 6 carbon atoms (i.e. C3, C4, C5 and C6), preferably between 3 and 4 carbon atoms (i.e. C3 and C4), preferably 3 or 4 carbon atoms (i.e. C3 or C4).

Preferably, the C3 to C6 olefinic feedstock comprises at least 20% by weight, preferably at least 50% by weight, preferably at least 90% by weight of olefins, preferentially at least 95% by weight of olefins, in particular at least 98% by weight of olefins or at least 99% by weight of olefins, relative to the total weight of the olefinic feedstock, relative to the total weight of the C3-C6 olefinic feedstock, the olefins containing between 3, 4, 5 and 6 carbon atoms, preferably 3 and/or 4 carbon atoms, said olefins preferably being mono-olefins.

The olefinic feedstock may optionally comprise paraffins, in particular paraffins of the different cuts at the limits of the initial and final distillation points of the feedstock under consideration, more particularly C3 to C6 paraffins, i.e. fully hydrogenated hydrocarbon compounds which are preferably aliphatic, preferably containing between 3 and 6 carbon atoms. Preferably, the olefinic feedstock may optionally comprise up to 80% by weight, preferably up to 50% by weight, more preferably up to 10% by weight, preferentially up to 5% by weight, in particular up to 2% or even up to 1% by weight of paraffins, relative to the total weight of the olefinic feedstock relative to the total weight of the C3 to C6 olefinic feedstock, preferably C3 and/or C4. Very preferably, the olefinic feedstock is free of paraffins, i.e. it comprises less than 0.5% by weight of paraffins, and preferably less than 0.1% by weight of paraffins, relative to the total weight of the olefinic feedstock. Processing an olefinic feedstock containing a low paraffin content, in particular containing paraffins in a content of less than or equal to 10% by weight, preferably less than or equal to 5% by weight, preferably less than or equal to 2% by weight, or even an olefinic feedstock free of paraffins, allows the oligomerization step to be performed at low pressure, in particular lower than that conventionally used for conventional (or fossil) feedstocks which generally comprise paraffin contents of more than 10% by weight and often between 40% and 80% by weight of paraffins.

In particular, the preferred olefinic feedstocks predominantly contain propylene and/or butenes and/or pentenes, preferably propylene and/or butenes (isobutene and/or n-butenes). The term “predominantly” is understood to mean at least 80% by weight, preferentially at least 90% by weight, relative to the total weight of the olefinic feedstock.

An olefinic feedstock that is particularly suitable for the process according to the invention is an olefinic feedstock essentially of C3 and C4, preferably of C3 or C4, i.e. at least 90% by weight, preferentially at least 95% by weight, preferably at least 98% by weight of C3 and/or C4 olefins relative to the total weight of the olefins contained in the olefinic feedstock. Thus, the olefinic feedstock advantageously comprises at least 85% by weight of propylene and/or butenes (in particular isobutene and/or n-butenes), preferably at least 90% by weight, preferentially at least 95% by weight, preferably at least 98% by weight of propylene and/or butenes relative to the total weight of the olefinic feedstock. The olefinic feedstock may in particular be chosen from a “polymer grade” propylene feedstock, a feedstock comprising essentially propylene (i.e. at least 90% by weight of propylene) and a small amount of butenes (i.e. less than 10% by weight of butenes), a feedstock consisting essentially of isobutene (i.e. at least 90% by weight of isobutene), a feedstock consisting essentially of n-butenes (but-1-ene and but-2-ene) (i.e. at least 90% by weight of but-1-ene and but-2-ene), and mixtures thereof.

In the preferred embodiment of the invention in which the olefinic feedstock is a C3 and/or C4 olefinic feedstock, said olefinic feedstock may also contain C5 and/or C6 compounds. In this case, the content of C5 and/or C6 compounds is preferably less than or equal to 5% by weight in the olefinic feedstock, preferably less than or equal to 2% by weight, or even preferably less than or equal to 1% by weight relative to the weight of the olefinic feedstock. In this case, the content of C5 and/or C6 olefins in the olefinic feedstock is preferably less than 0.8% by weight, preferably less than 0.6% by weight. Optionally, the olefinic feedstock of this preferred embodiment may also comprise paraffins, in particular propane and/or butane. Preferably, the C3 and/or C4 olefinic feedstock is free of propane and/or butane (i.e. it comprises less than 0.5% by weight of paraffins, and preferably less than 0.1% by weight of paraffins, relative to the total weight of the olefinic feedstock), thus enabling the oligomerization step to be performed at a lower pressure relative to the oligomerization of a feedstock comprising paraffins. Preferably, the olefinic feedstock is free of C5 and/or C6 paraffins, i.e. it comprises less than 0.5% by weight, preferably less than 0.1% by weight of C5 and/or C6 paraffins, so as to limit the amount of inert compounds introduced notably into step a).

A preferred olefinic feedstock is an olefinic C4 cut, which comprises at least 98% by weight of 1-butene and 2-butene n-butenes, preferably less than 2% of n-butane, and preferably less than 0.5% of n-butane, the percentages being given relative to the total weight of the olefinic feedstock.

Another preferred olefinic feedstock is an olefinic C4 cut, which comprises in particular at least 90% by weight of isobutene, or even at least 92% by weight of isobutene, and in particular at most 97% by weight of isobutene, and optionally butane and/or isobutane and/or n-butenes, in particular between 3% and 10% by weight of butane and/or isobutane and/or n-butenes, the percentages being given relative to the total weight of the olefinic feedstock.

The olefinic feedstock may also be an olefinic C3-C4 cut (i.e. comprising propylene and butenes), for example comprising at least 90% by weight of propylene and up to 10% by weight of butenes, said feedstock preferably being free of paraffins (i.e. comprising less than 0.5% by weight of paraffins, preferably less than 0.1% by weight of paraffins), the percentages being given relative to the total weight of the olefinic feedstock.

Another preferred olefinic feedstock is an olefinic C3 cut, preferably comprising at least 90% by weight of propylene, preferably at least 98% by weight of propylene. It may comprise propane, preferentially up to 2% by weight of propane.

Preferably, the olefinic feedstock is at least partly, very advantageously entirely, biobased, so as to produce biobased upgradable products. The C3 to C6 olefinic feedstock, preferably the C3 and/or C4 olefinic feedstock, may notably be derived from a Fischer-Tropsch unit, a unit for the production of olefins from methanol and/or a unit for the dehydration of alcohols, for example isobutanol, in particular derived from biomass, for example derived from the fermentation of sugars.

The olefinic feedstock may also come from a conventional unit. In this case, it is preferably used as a mixture with feedstocks of biobased origin, preferably in weight ratios between a conventional olefinic feedstock and a biobased olefinic feedstock of between 90/10 and 10/90, preferably between 80/20 and 20/80. Preferably, a conventional olefinic feedstock comes from a steam cracking unit, an FCC catalytic cracking unit, a selective diolefin hydrogenation unit (known as SHU), or a paraffin dehydrogenation unit, either pure or as a mixture, and/or from any other unit leading to the production of light olefins.

The olefinic feedstock treated in the process according to the invention can advantageously undergo a pretreatment step before being sent to the oligomerization step a). Such a pretreatment step makes it possible to eliminate any compound which may cause poisoning of the oligomerization catalysts, notably basic nitrogen compounds, water, sulfur derivatives and basic nitrogen derivatives.

Preferably, the olefinic feedstock is free of sulfur or sulfur compounds, i.e. it comprises a content of less than or equal to 20 ppm by weight, preferably less than or equal to 12 ppm by weight, preferentially less than or equal to 10 ppm by weight of sulfur relative to the weight of the olefinic feedstock, making it possible to avoid or at least limit poisoning of the oligomerization catalyst of step a). If the olefinic feedstock contains sulfur (i.e. more than 20 ppm by weight), the process advantageously comprises a step of pretreatment of the olefinic feedstock, located upstream of the oligomerization step a), preferably involving an adsorption section and/or a water washing section and/or a dedicated hydrotreating section, thus making it possible to protect the oligomerization catalyst of step a).

Preferably, the feedstock of the process according to the invention is free of nitrogen or nitrogen compounds, i.e. it comprises a content of less than or equal to 0.1 ppm by weight of nitrogen element relative to the total weight of the olefinic feedstock, thus avoiding or at least limiting the poisoning of the oligomerization catalyst of step a). If the olefinic feedstock contains nitrogen, the process advantageously comprises a pretreatment step of the olefinic feedstock, located upstream of the oligomerization step a), preferably involving an adsorption and/or water washing and/or hydrotreating section, thus makings it possible to protect the oligomerization catalyst of step a).

Preferably, the olefinic feedstock treated by means of the process according to the invention is free of butadiene, in particular 1,3-butadiene, i.e. it comprises a content of less than or equal to 0.1% by weight, preferably less than or equal to 500 ppm by weight of butadiene, in particular of 1,3-butadiene, relative to the total weight of the olefinic feedstock, thus protecting the oligomerization catalyst. If the olefinic feedstock contains butadiene, in particular 1,3-butadiene, the process advantageously comprises a step of pretreatment of the olefinic feedstock, located upstream of the oligomerization step a), preferably involving a selective hydrogenation section.

According to a particular embodiment of the invention, the process may comprise an optional olefinic feedstock separation step, advantageously located upstream of the oligomerization step a), so as to at least partially separate the C5 and C6 compounds present in said olefinic feedstock. This optional separation step thus makes it possible to produce a fraction comprising the C5 and C6 compounds possibly present in the feedstock and at least one fraction comprising the C3 and C4 compounds. A person skilled in the art can regulate the separation so as to push the fractionation more or less and to separate the fraction comprising C3 and C4 compounds into a C3 fraction (in particular enriched in C3, more particularly propylene) and a C4 fraction (in particular enriched in C4, more particularly isobutene and/or n-butenes). Thus, this optional separation step makes it possible to concentrate the feedstock in particular in C3 and/or C4 olefinic compounds. The fraction comprising the C3 and C4 compounds, the C3 fraction or the C4 fraction is then advantageously sent to the oligomerization step a), preferably directly. The fraction comprising the C5 and C6 compounds can, for its part, be purged and upgraded, for example by being integrated into a gasoline pool. According to a most particular embodiment of the invention, the process may comprise an optional olefinic feedstock separation step, located upstream of the oligomerization step a), to separate at least a C3 fraction and a C4 fraction, the two fractions, the C3 fraction and the C4 fraction, each undergoing steps a), b) and c) and optionally d) in parallel, and the heavy fractions being able to be remixed at the end of step c) or optionally at the end of the hydrogenation step d).

### Oligomerization Step a)

The process according to the invention comprises an oligomerization step, more particularly involving a heterogeneous oligomerization reaction (or called by heterogeneous catalysis), performed in the presence of at least one oligomerization catalyst, to produce a reaction effluent comprising dimers, trimers and oligomers. Specifically, this oligomerization step a) makes it possible to obtain a hydrocarbon mixture containing monoolefins with a number of carbon atoms predominantly greater than or equal to 6, preferably greater than or equal to 8, preferably greater than or equal to 9, the term “predominantly” meaning here at least 90% by weight of C6+ hydrocarbons, preferably C8+, preferably C9+, relative to the weight of the hydrocarbon mixture obtained. The hydrocarbon mixture obtained may also comprise unreacted C3 to C6 feedstock olefins.

According to the invention, the oligomerization step a) is fed with at least the olefinic feedstock, optionally pretreated and/or optionally separated. Preferably, the oligomerization step a) is also fed with a first recycle comprising, preferably consisting of, at least part of the light fraction from step b); said first recycle is advantageously prepared and then transferred to step a) in step c). Preferably, the oligomerization step a) is also fed with a second recycle comprising, preferably consisting of, at least part of the intermediate fraction obtained from step b); said first recycle is advantageously prepared and then transferred to step a) in step c).

Advantageously, the oligomerization step a) is performed, preferably in the liquid phase (i.e. the olefinic feedstock and the products formed are in liquid form under the temperature and pressure conditions used), in the presence of an oligomerization catalyst, which is preferably solid. Preferably, the oligomerization step a) is performed at a temperature of between 2° and 500° C., at a pressure of between 1.0 and 10 MPa, and with an HSV preferably between 0.1 and 0.5 h 1, preferably between 0.2 and 0.3 h¹. The HSV (or hourly space velocity) is, according to the invention, defined by the ratio between the volume flow rate of fresh olefinic feedstock in particular at 15° C. and 1 atm and the volume of oligomerization catalyst in particular in operation (also called in use). The temperature at which the oligomerization step a) is performed, between 2° and 500° C., advantageously corresponds to the temperature at the inlet of step a), preferably at the inlet of the reactor used in step a). The operating conditions of temperature, pressure and hourly space velocity may be adjusted by a person skilled in the art in particular as a function of the composition of the olefinic feedstock and the nature of the oligomerization catalyst used, so as to maximize the yields of middle distillates, in particular the yields of kerosene or gas oil.

Advantageously, step a) uses at least one oligomerization catalyst, preferably between one and three different oligomerization catalysts, preferably one oligomerization catalyst. Any type of oligomerization catalyst known to those skilled in the art may be used as oligomerization catalyst in step a). More particularly, the oligomerization catalyst(s) of step a) may be any type of acid catalyst, in particular chosen from silica-impregnated phosphoric acid-based catalysts (supported phosphoric acid, of “SPA” type), ion-exchange resins, silica-alumina and pure zeolites or zeolites supported on an alumina support. Preferably, the oligomerization catalyst(s) of step a) are chosen from ion-exchange resins, preferably cation-exchange resins, silica-alumina (i.e. comprising silica and alumina) and pure zeolites or zeolites supported on an alumina support.

When the oligomerization catalyst is chosen from the SPA-type catalysts, step a) is preferably performed at a temperature, advantageously the inlet temperature, of between 10° and 300° C., preferentially between 16° and 250° C., and at a pressure preferably between 1.5 and 6.5 MPa, preferentially between 1.5 and 4.0 MPa.

Zeolite-based catalysts are particularly suitable for producing linear or sparingly-branched heavy olefins, which in particular allow the production of high-quality gas oil, i.e. after hydrogenation, a gas oil having a cetane number of greater than 45. When the oligomerization catalyst is chosen from the zeolite-based catalysts, step a) is preferably performed at a temperature, advantageously the inlet temperature, of between 15° and 500° C., preferentially between 20° and 350° C., at a pressure preferably between 2.0 and 10.0 MPa, preferentially between 3.0 and 6.5 MPa. Preferably, the zeolite-based oligomerization catalyst comprises at least one zeolite chosen from the group consisting of aluminosilicate-type zeolites having an overall Si/Al atomic ratio of more than 10 and an 8, 10 or 12MR pore structure. Said zeolite is more preferably selected from the group consisting of zeolites with a structural type of ferrierite, chabazite, Y and US-Y, ZSM-5, ZSM-12, NU-86, mordenite, ZSM-22, NU-10, ZBM-30, ZSM-11, ZSM-57, ZSM-35, IZM-2, ITQ-6 and IM-5 and SAPO, and mixtures thereof. Very preferably, said zeolite is selected from the group consisting of ferrierite, ZSM-5, mordenite and ZSM-22 zeolites, and mixtures thereof. Even more preferably, the zeolite used is ZSM-5.

Ion-exchange resin type catalysts are chosen for their good mechanical strength in the temperature and pressure ranges used in step a). When the oligomerization catalyst is chosen from ion-exchange resins, step a) is preferably performed at a temperature, advantageously at the inlet temperature, of between 20° C. and 250° C., preferentially between 70° C. and 180° C., and at a pressure preferably between 2.0 and 10.0 MPa, preferentially between 3.0 and 6.5 MPa. Ion-exchange resin catalysts, which are inexpensive and non-regenerable, have the advantage of having acceptable cycle times in a fixed bed operation since they are less sensitive to contaminants than zeolites and silica-alumina. Very preferably, the ion-exchange resin catalyst used in step a) is a copolymer of monovinyl aromatics and of polyvinyl aromatics, preferably a copolymer of divinylbenzene and of styrene, which is preferably sulfonated, notably having a degree of crosslinking of between 20% and 45%, preferably between 30% and 40%, and preferably equal to 35%, and an acid strength, representing the number of active sites in said resin, determined by assay, preferably by conductimetry, of the H⁺ ions released by the acidic resin after exchange with Na⁺ ions (cf. ASTM D4266), of between 1 and 10 mmol H⁺ equivalent per gram, and preferably of between 3.5 and 6 mmol H⁺ equivalent per gram. For example, the acidic oligomerization catalyst, of the ion-exchange resin type, used in step a), is a commercial acidic resin sold under the reference TA801 by the company Axens.

Silica-alumina type catalysts have the advantage of being regenerable so that, despite their cost which is higher than the one of resins, substantial savings are made in terms of catalyst consumption. When the oligomerization catalyst is chosen from silica-aluminas, step a) is preferably performed at a temperature of between 2° and 300° C., preferentially between 3° and 220° C., preferably between 4° and 200° C., and at a pressure preferably between 1.5 and 6.5 MPa, preferentially between 2.0 and 4.0 MPa. The temperature at which the oligomerization step a) in the presence of silica-alumina is performed, preferably between 2° and 300° C., preferentially between 3° and 220° C., preferably between 4° and 200° C., advantageously corresponds to the temperature at the inlet of step a), preferably at the inlet of the reactor used in step a). The silica-alumina-based oligomerization catalyst(s) are one or more amorphous catalysts preferably consisting of an amorphous mineral material chosen from silica-aluminas and silica-treated aluminas, and preferably from silica-aluminas. In the silica-alumina-based oligomerization catalyst used in step a), the SiO₂/Al₂O₃ mass ratio is between 0.1 and 10. Preferentially, the silica-alumina present in the oligomerization catalyst used in oligomerization step a) has the following characteristics:
a silica (SiO₂) mass content of between 5% and 95% by weight, preferably of between 10% and 80% by weight, more preferably of between 20% and 80% by weight and even more preferably of between 25% and 75% by weight, relative to the weight of the silica-alumina present in the oligomerization catalyst;
a content of cationic impurities advantageously less than 0.1% by weight, preferably less than 0.05% by weight and even more preferably less than 0.025% by weight, relative to the weight of the silica-alumina present in the oligomerization catalyst, the content of cationic impurities being the total content of alkalis, in particular of sodium.

The catalysts prepared as described in patent FR 2 926 812 may be suitable as oligomerization catalyst for step a).

According to a particular embodiment of the invention, the oligomerization catalyst used in step a) consists entirely of silica-alumina, i.e. it is free of any other element (i.e. it comprises less than 0.5% by weight, preferably less than 0.1% by weight of any element other than silica and alumina).

According to another particular embodiment of the invention, the oligomerization catalyst used in step a) may contain at least one metal element chosen from the metals of groups IVB, VB, VIB and VIII. Among the metals of group IVB, titanium, zirconium and/or hafnium may be present in the oligomerization catalyst. Among the metals of group VB, vanadium, niobium and/or tantalum may be present in the oligomerization catalyst. Among the metals of group VIB, chromium, molybdenum and/or tungsten may be present in the oligomerization catalyst. Among the metals of group VIII, metals belonging to the first line of the group VIII metals, namely iron, cobalt and nickel, are preferred. The content of these metals may be up to 10% by weight, relative to the weight of the oligomerization catalyst. The oligomerization catalyst may optionally also contain silicon as a doping element deposited on the silica-alumina.

Very advantageously, the oligomerization step a) is performed in the presence of a silica-alumina-based catalyst at a temperature, advantageously at the inlet of step a), of between 20° C. and 300° C., preferentially between 25 and 220° C., preferably between 30° C. and 200° C., and at a pressure of between 1.5 and 6.5 MPa, preferentially from 2.0 to 4.0 MPa. According to a particular embodiment, when the olefinic feedstock is a C3 and/or C4 olefinic feedstock, the oligomerization step a) is preferably performed in the presence of a silica-alumina catalyst, at a temperature, advantageously at the inlet of step a), preferably between 25 and 200° C., preferentially between 3° and 190° C., and a pressure between 1.5 and 6.5 MPa, preferably between 2.0 and 4.0 MPa, preferentially to produce kerosene. According to another particular embodiment, when the olefinic feedstock is a C3 and/or C4 olefinic feedstock, the oligomerization step a) is preferably performed in the presence of a silica-alumina catalyst, at a temperature, advantageously at the inlet of step a), preferably between 35 and 200° C., preferentially between 4° and 190° C., and a pressure between 1.5 and 6.5 MPa, preferentially between 2.0 and 4.0 MPa, preferentially to produce gas oil.

Preferably, the oligomerization catalyst(s) of oligomerization step a) are in the form of spheres, pellets or extrudates, preferentially extrudates. Very advantageously, the oligomerization catalyst(s) are in the form of extrudates with a diameter of between 0.5 and 5 mm and more particularly between 0.7 and 2.5 mm. The forms of the extrudates are cylindrical (which may or may not be hollow), twisted cylindrical, multilobal (for example 2, 3, 4 or 5 lobes) or annular. The cylindrical and multilobal forms are preferably used, but any other form may be used. In a very particular embodiment of the invention, the oligomerization step is performed in the presence of a silica-alumina-based oligomerization catalyst, preferably consisting of silica-alumina, which is in the form of trilobal extrudates.

Advantageously, the oligomerization step may use one or more reactors, preferably at least two, preferably at least three reactors, and up to ten, preferably six reactors, arranged in parallel or in series, preferably in series, comprising one or more different oligomerization catalysts, preferably comprising the same oligomerization catalyst. The operating conditions and also the oligomerization catalysts described above can be applied to any of the reactors. Very particularly, the oligomerization step involves at least two or even three reactors in series. To ensure continuous operation of the oligomerization step, it is possible to have at least two reactors or reactor trains, one of the reactors (or one of the reactor trains) being in the reaction phase, the other reactor (or one of the reactor trains) being in the regeneration phase, if the level of impurities in the feedstock leads to rapid deactivation of the catalyst. Optionally, the oligomerization step may also involve heat exchangers upstream of the reactor(s) to heat the olefinic feedstock.

The oligomerization reaction is exothermic. At least part of the temperature increase related to the exothermicity of the oligomerization reaction can be controlled by the first and second recycles (comprising, respectively, at least part of the light fraction corresponding at least in part to the unconverted feedstock and at least in part to the intermediate fraction), introduced into oligomerization step a). The exothermicity may also be controlled at least in part by diluting the olefinic feedstock by adding paraffins from a source external to the process, said paraffins being of the same molecular weight and/or heavier than the olefinic feedstock, said paraffins being aliphatic or cyclic, and/or by introducing a stream of inerts corresponding to a part of the hydrogenated heavy fraction obtained at the end of step d), and in particular to a part of the kerosene and/or gas oil cut possibly separated out in the optional step e) or a mixture of the kerosene and/or gas oil cuts and the residue of the optional step e). In the latter case, the inert stream is preferably between 0 and 6 times the fresh olefinic feedstock by weight, preferably between 0.5 and 4 by weight.

The oligomerization step a) thus produces a reaction effluent which comprises dimers, trimers and oligomers. This reaction effluent is totally or partially sent to a fractionation step b).

### Fractionation Step b)

The process according to the invention comprises a step of fractionating the reaction effluent obtained at the end of step a), into at least:
a light fraction comprising at least part of the olefinic feedstock not converted in step a);
an intermediate fraction comprising at least part, preferably all, of the dimers and trimers advantageously produced in step a); and
a heavy fraction, comprising the oligomers present in the reaction effluent from step a), in particular olefinic compounds containing between 6 and 30, preferably between 9 and 25 carbon atoms, and more particularly between 9 and 16 carbon atoms in the case of kerosene or between 10 and 25 carbon atoms in the case of gas oil.

The light fraction comprises, preferably consists of, at least part, preferably all, of the olefinic feedstock not converted in step a). It thus advantageously comprises C3 to C6 olefins, preferably C3 and/or C4 olefins, which were not converted in step a). The light fraction may optionally also comprise unreacted, i.e. non-olefinic, compounds, in particular paraffins that are notably already present in the fresh olefinic feedstock. The amount of this light fraction depends in particular on the conversion of the olefinic feedstock per pass of the oligomerization step a). This fraction is advantageously totally or partially recycled into step a); indeed, it constitutes at least part of the first recycle prepared in step c). Optionally, part of this light fraction can be purged, continuously or discontinuously, in particular when the olefinic feedstock contains compounds which do not oligomerize, for instance paraffins. In case of purging, the purged stream can be upgraded to LPG (liquefied petroleum gas), for example. Optionally, the light fraction may comprise C1 to C2 compounds, possibly generated during step a) and resulting from cracking and recombination reactions.

Optionally, at the end of the fractionation step, a gaseous fraction may also be separated out, the gaseous fraction which comprises C1 to C2 compounds, optionally generated during step a) and resulting from cracking and recombination reactions. The gaseous fraction possibly separated out in step b) is preferably purged (i.e. taken out of the process) in a continuous or discontinuous manner, for example to be upgraded.

The intermediate fraction comprises, preferably consists of, at least part, preferably all, of the dimers and trimers advantageously produced in step a). The products contained therein, in particular the dimers and trimers, are too light to be upgraded to middle distillates, in particular to kerosene or gas oil. The intermediate fraction may also optionally contain unconverted C5 to C6 olefins if the olefin feedstock contains C5 to C6 olefins. The intermediate fraction may also contain paraffins from the olefinic feedstock boiling in the same ranges as the intermediate fraction. Preferably, the intermediate fraction is free of paraffins, i.e. it comprises less than 0.5% by weight, preferably less than 0.1% by weight of paraffins relative to the total weight of the intermediate fraction. Preferably, the intermediate fraction comprises C5+ olefin oligomers and preferably has a T95 of less than 170° C., in particular less than 140° C. in the case of kerosene production or less than 165° C., preferably less than 170° C. in the case of gas oil production. It can therefore also be called C5-140° C. or C5-165° C. (preferably C5-170° C.). Optionally, at least part of the intermediate fraction can be recovered and taken out of the process (i.e. purged) to be treated or directly upgraded, in particular as gasoline. This optional purged part of the intermediate fraction may undergo a hydrogenation step, and notably may be sent to step d) of the process or to a hydrogenation step distinct from the process according to the invention and operated, for example, under conditions similar to those described for step d), before being integrated into a gasoline pool.

The heavy fraction advantageously comprises the oligomers present in the reaction effluent from step a). Advantageously, it comprises in particular olefinic compounds containing between 6 and 30, preferably between 9 and 25 carbon atoms. Preferably, the heavy fraction has a T5 of greater than or equal to 140° C., in particular greater than or equal to 140° C. in the case of kerosene production, or greater than or equal to 165° C., preferably greater than or equal to 170° C., in the case of gas oil production. Preferably, the heavy fraction is composed of C9+ olefinic oligomers and very preferably boils between 14° and 300° C. (also called 140-300° C. fraction) or at a temperature greater than or equal to 165° C., preferably greater than or equal to 170° C. According to a preferred embodiment of the invention, the heavy fraction may correspond to a kerosene fraction with a cutting point making it possible to reach a flash point greater than or equal to 38° C. According to another preferred embodiment of the invention, the heavy fraction may correspond to a gas oil fraction with a cutting point allowing a flash point of greater than or equal to 55° C. to be reached.

Advantageously, the fractionation step b) uses one or more distillation columns, preferably between one and three distillation columns.

### Recycle Step c)

The recycle step c) of the process according to the invention involves:
preparing a first recycle which comprises, preferably consists of, at least part of the light fraction,
preparing a second recycle which comprises, preferably consists of, at least part of the intermediate fraction, from the fractionation step b),
transferring the first recycle to the oligomerization step a), and
transferring the second recycle to step a).

Advantageously, all or part of the light fraction from the separation step b) constitutes the first recycle which is then recycled to the oligomerization step a), preferably directly at the inlet of step a) and preferably upstream of any exchangers used in step a) upstream of the reactors to heat the olefinic feedstock. The first recycle advantageously makes it possible to maximize the overall conversion and also to manage at least part of the exothermicity of the oligomerization reaction in step a). Preferably, the first recycle, which corresponds to the part of the light fraction recycled to a), represents an amount such that the weight ratio between the first recycle and the olefinic feedstock, which feed the oligomerization step a), is between 0.3 and 1.5, preferably between 0.5 and 1.2.

Advantageously, all or part of the intermediate fraction from the separation step b) constitutes the second recycle which is then recycled to step a), preferably directly and in particular upstream of any exchangers used upstream of the reactors in step a). Preferably, the intermediate fraction is not cooled before being transferred totally or partially, as second recycle, to the oligomerization step a), which participates notably towards the preheating of the olefinic feedstock at the inlet of step a) by simple mixing. Preferably, the second recycle, which corresponds to the part of the intermediate fraction recycled to a), represents an amount such that the weight ratio between the second recycle and the olefinic feedstock, which feed the oligomerization step a), is between 0.5 and 10.0, preferentially between 1.0 and 5.0, preferably between 1.0 and 4.0.

The recycling of at least part of these two fractions, the light fraction and the intermediate fraction, in particular the recycling of at least part of the intermediate fraction, makes it possible to increase the overall conversion and to maximize the yield of the target products, in particular middle distillates, more particularly kerosene or gas oil, by strongly favouring the selectivity towards the target products, in particular kerosene or gas oil.

Preferably, the heavy fraction which comprises oligomers and which comes from fractionation step b) is not recycled, and in particular is not recycled to oligomerization step a). Indeed, the heavy fraction comprises heavy olefinic compounds containing in particular between 6 and 30, preferably between 9 and 25 carbon atoms. These compounds are known to have reactivity which is not negligible with respect to oligomerization. Thus, if such olefinic compounds were recycled to oligomerization step a), highly heavy compounds which are not desired would be formed, which decreases selectivity and hence decreases middle distillates yields. In a preferred manner, the process for preparing middle distillates according to the invention thus lacks recycling of the heavy fraction obtained from step b). In particular, in the case of kerosene production, the process advantageously lacks recycling of the heavy fraction of which T5 is greater than or equal to 140° C. In the case of gas oil production, the process advantageously lacks recycling of the heavy fraction of which T5 is greater than or equal to 165° C., preferably greater than or equal to 170° C.

According to a first particular embodiment of the invention, the olefinic feedstock is essentially composed of isobutene, preferably at least 90% by weight of isobutene and very advantageously less than 0.5% by weight, or even less than 0.1% by weight of paraffins such as butane, relative to the total weight of the olefinic feedstock, and the oligomerization step a) is advantageously performed in the presence of silica-alumina, preferably at a temperature of between 2° and 100° C., preferentially between 3° and 90° C. and very preferentially between 35° C. and 85° C., at a pressure preferentially between 1.5 and 6.5 MPa, preferably between 2.0 and 4.0 MPa, and an HSV preferably between 0.20 and 0.30 h 1, preferentially between 0.20 and 0.25 h 1. In this embodiment, a stream of inerts (i.e. a stream of compounds that are inert with respect to the oligomerization reaction, i.e. which do not react under the operating conditions of step a)), preferably composed at least partly of a portion of the hydrogenated heavy fraction obtained at the end of the hydrogenation step d), is preferably used to feed step a) so as to control the exothermicity of the oligomerization reaction and thus the reactivity. According to this first particular embodiment, the intermediate fraction separated out in step b) advantageously has a T95 of less than 140° C.; the heavy fraction has a T5 greater than or equal to 140° C. and preferably boils between 14° and 300° C. According to this first particular embodiment, the second recycle prepared in step c) and which preferably consists of at least part, preferably all, of the intermediate fraction separated out in b), represents an amount by weight such that the weight ratio between the second recycle and the olefinic feedstock at the inlet of step a) is between 1.0 and 5.0, preferably between 1.5 and 2.5, the second recycle being sent to the oligomerization step a).

According to a second particular embodiment of the invention, the olefinic feedstock is composed essentially of propylene, preferably at least 90% by weight of propylene, optionally up to 10% by weight of butenes and very advantageously less than 0.5% by weight, or even less than 0.1% by weight of paraffins, relative to the total weight of the olefinic feedstock and the oligomerization step a) is advantageously performed in the presence of silica-alumina, preferably at a temperature of between 10° and 180° C., preferentially between 11° and 170° C., preferably between 115 and 165° C., at a pressure preferentially between 1.5 and 6.5 MPa, preferably between 2.0 and 4.0 MPa, and an HSV preferably between 0.20 and 0.30 h 1, preferentially between 0.20 and 0.25 h 1. In this particular embodiment, a stream of inerts (i.e. a stream of compounds that are inert with respect to the oligomerization reaction, i.e. which do not react under the operating conditions of step a)), preferably consisting at least in part of a portion of the hydrogenated heavy fraction obtained at the end of the hydrogenation step d), is preferably used to feed step a) so as to control the exothermicity of the oligomerization reaction and thus the reactivity. According to this second particular embodiment, the intermediate fraction separated out in step b) advantageously has a T95 of less than 140° C.; the heavy fraction has a T5 greater than or equal to 140° C. and preferably boils between 14° and 300° C. According to this second particular embodiment, the second recycle prepared in step c) and which preferably consists of at least part, preferably all, of the intermediate fraction separated out in b), represents an amount by weight such that the weight ratio between the second recycle and the olefinic feedstock at the inlet of step a) is between 1.0 and 5.0, preferably between 1.5 and 2.0, the second recycle being sent to the oligomerization step a).

According to a third particular embodiment of the invention, the olefinic feedstock is essentially composed of n-butenes (but-1-enes and but-2-enes), preferably of at least 98% by weight of butenes and very advantageously less than 0.5% by weight of paraffins such as butane, relative to the total weight of the olefinic feedstock, and the oligomerization step a) is advantageously performed in the presence of silica-alumina, preferably at a temperature between 13° and 200° C., preferentially between 14° and 190° C., preferably between 145 and 185° C., at a pressure preferentially between 1.5 and 6.5 MPa, preferably between 2.0 and 4.0 MPa, and an HSV preferably between 0.20 and 0.30 h 1, preferentially between 0.20 and 0.25 h 1. Optionally, a stream of inerts (i.e. a stream of compounds that are inert with respect to the oligomerization reaction, i.e. which do not react under the operating conditions of step a)), preferably composed at least partly of a portion of the hydrogenated heavy fraction obtained at the end of the hydrogenation step d), may be used to feed step a) so as to control the exothermicity of the oligomerization reaction and thus the reactivity. According to this third particular embodiment, the intermediate fraction separated out in step b) advantageously has a T95 of less than 140° C.; the heavy fraction has a T5 greater than or equal to 140° C. and preferably boils between 14° and 300° C. According to this third particular embodiment, the second recycle prepared in step c) and which preferably consists of at least part, preferably all, of the intermediate fraction separated out in b), represents an amount by weight such that the weight ratio between the second recycle and the olefinic feedstock at the inlet of step a) is between 1.0 and 5.0, preferably between 3.5 and 4.0, the second recycle being sent to the oligomerization step a).

According to a fourth particular embodiment of the invention, the olefinic feedstock is essentially composed of isobutene, preferably at least 90% by weight of isobutene and very advantageously less than 0.5% by weight, or even less than 0.1% by weight of paraffins such as butane, relative to the total weight of the olefinic feedstock, and the oligomerization step a) is advantageously performed in the presence of silica-alumina, preferably at a temperature of between 3° and 100° C., preferentially between 4° and 90° C., at a pressure preferentially between 1.5 and 6.5 MPa, preferably between 2.0 and 4.0 MPa, and an HSV of preferably between 0.20 and 0.30 h 1, preferentially between 0.25 and 0.30 h 1. In this embodiment, a stream of inerts, preferably composed at least partly of a portion of the hydrogenated heavy fraction obtained at the end of hydrogenation step d), is preferably used to feed step a) so as to control the exothermicity of the oligomerization reaction. According to this fourth particular embodiment, the intermediate fraction separated in step b) advantageously has a T95 of less than 165° C.; the heavy fraction has a T5 greater than or equal to 165° C. According to this fourth particular embodiment, the second recycle, which preferably consists of at least part, preferably all, of the intermediate fraction separated out in b), represents an amount by weight such that the weight ratio between the second recycle and the olefinic feedstock at the inlet of step a) is between 1.0 and 4.0, preferably between 1.0 and 2.0, the second recycle being sent to the oligomerization step a).

According to a fifth particular embodiment of the invention, the olefinic feedstock is composed essentially of propylene, preferably of at least 90% by weight of propylene, optionally up to 10% by weight of butenes and very advantageously less than 0.5% by weight, or even less than 0.1% by weight of paraffins, relative to the total weight of the olefinic feedstock, and the oligomerization step a) is advantageously performed in the presence of silica-alumina, preferably at a temperature of between 11° and 180° C., preferentially between 12° and 170° C., at a pressure preferentially between 1.5 and 6.5 MPa, preferably between 2.0 and 4.0 MPa, and an HSV preferably between 0.20 and 0.30 h 1, preferentially between 0.25 and 0.30 h 1. In this embodiment, a stream of inerts, preferably composed at least partly of a portion of the hydrogenated heavy fraction obtained at the end of hydrogenation step d), is preferably used to feed step a) so as to control the exothermicity of the oligomerization reaction. According to this fifth particular embodiment, the intermediate fraction separated in step b) advantageously has a T95 of less than 165° C.; the heavy fraction has a T5 greater than or equal to 165° C. According to this fifth particular embodiment, the second recycle, which preferably consists of at least part, preferably all, of the intermediate fraction separated out in b), represents an amount by weight such that the weight ratio between the second recycle and the olefinic feedstock at the inlet of step a) is between 1.0 and 4.0, preferably between 1.0 and 1.5, the second recycle being sent to the oligomerization step a).

According to another particular embodiment of the invention, the olefinic feedstock is essentially composed of n-butenes (but-1-enes and but-2-enes), preferably of at least 98% by weight of butenes and very advantageously less than 0.5% by weight of paraffins such as butane, relative to the total weight of the olefinic feedstock, and the oligomerization step a) is advantageously performed in the presence of silica-alumina, preferably at a temperature between 14° and 200° C., preferentially between 145 and 190° C., at a pressure preferentially between 1.5 and 6.5 MPa, preferably between 2.0 and 4.0 MPa, and an HSV preferably between 0.20 and 0.30 h 1, preferentially between 0.25 and 0.30 h 1. In this embodiment, a stream of inerts, preferably composed at least partly of a portion of the hydrogenated heavy fraction obtained at the end of hydrogenation step d), is preferably used to feed step a) so as to control the exothermicity of the oligomerization reaction. According to this particular embodiment, the intermediate fraction separated in step b) advantageously has a T95 of less than 165° C.; the heavy fraction has a T5 greater than or equal to 165° C. According to this particular embodiment, the second recycle, which preferably consists of at least part, preferably all, of the intermediate fraction separated out in b), represents an amount by weight such that the weight ratio between the second recycle and the olefinic feedstock at the inlet of step a) is between 1.0 and 4.0, preferably between 3.0 and 3.5, the second recycle being sent to the oligomerization step a).

In these six particular embodiments of the invention, the temperature at which the oligomerization step a) in the presence of silica-alumina is performed advantageously corresponds to the temperature at the inlet of step a), preferably at the inlet of the reactor used in step a).

### Hydrogenation Step d)

The process according to the invention comprises a step of hydrogenation of at least part, preferably all, of the heavy fraction separated out in step b) in the presence of hydrogen, to obtain a hydrogenated heavy fraction.

The hydrogenation step enables the olefinic bonds of at least part, preferably all, of the heavy fraction from step c) to be saturated, so as to produce paraffins which can be incorporated directly into fuel pools, in particular the kerosene pool (or jet pool) and in a very particular manner into the SPK (“Synthetic Paraffinic Kerosene”) jet pool which meets the specifications of the standard ASTM D7566, Appendix 5, or the gas oil pool and in a very particular manner into the gas oil pool which meets the specifications of the European standard 15940. Step d) of hydrogenating the unsaturated compounds in particular makes it possible to substantially improve the smoke point of the heavy fraction, and in particular of the middle distillates produced, and/or to remove any sulfur and/or nitrogen impurities.

Preferably, the hydrogenation step d) is performed in the presence of a catalyst preferentially comprising at least one group VIII metal, notably nickel, palladium or platinum, deposited on an inert support, for instance silica or alumina. Preferably, the hydrogenation step is performed in the presence of a palladium-based or nickel-based catalyst on an alumina support. Nevertheless, any other catalyst allowing the product of the oligomerization step to be hydrogenated, and in particular the heavy fraction with, in particular, C9+ olefins, may be used. For example, a catalyst chosen from catalysts such as NiMo, CoMo, or NiCoMo on alumina, and mixtures thereof, may be used.

The hydrogenation step d) is performed, preferably in the liquid phase, advantageously at a pressure of between 0.5 and 5.0 MPa, preferably between 1.0 and 5.0 MPa, and preferably at a temperature of between 5° and 300° C., preferentially between 6° and 200° C., in the presence of hydrogen, preferably at a content of between 0.5% and 3% by weight relative to the weight of the part of the heavy fraction feeding step d).

Preferably, in step d), a degree of hydrogenation of at least 90%, preferentially greater than or equal to 95%, preferably greater than or equal to 99%, is achieved.

Advantageously, the hydrogenated heavy fraction thus comprises, preferably consists of, at least in part of middle distillates, and more particularly of a kerosene cut very advantageously meeting the kerosene specifications of the standards in force, in particular the kerosene specifications of standard ASTM D7566, in particular of standard ASTM D7566 Appendix 5, and/or a gas oil cut very advantageously meeting the gas oil specifications of the standards in force, in particular the gas oil specifications of European standard 15940. The hydrogenated heavy fraction obtained at the end of step d) may optionally be totally or partially sent to an optional separation step e).

Optionally, part of the hydrogenated heavy fraction obtained at the end of step d) is separated out to form an inert stream which can then be recycled into step a) to help control the exothermicity of the oligomerization reaction in step a), in parallel with the first recycle. Preferably, the weight amount of the inert stream recycled into step a) is from 0 to 6 times, preferably from 0.5 to 4 times by weight of the weight of the fresh olefinic feedstock.

### Optional Separation Step e)

The process according to the invention comprises a step of separating out the hydrogenated heavy fraction, so as to obtain at least one middle distillate cut, notably at least one kerosene cut and/or a gas oil fraction and optionally a gasoline fraction.

Very particularly, a kerosene base is separated out in step e) and this kerosene base preferably has a final evaporation temperature of between 14° and 300° C. and advantageously a flash point of greater than or equal to 38° C.; the separated gas oil cut preferably has an evaporation temperature of greater than or equal to 165° C., preferably greater than or equal to 170° C., and advantageously a flash point of at least 55° C.

According to a particular embodiment, the optional separation step e) advantageously makes it possible to obtain:
a kerosene cut whose lower (initial) distillation point is preferably at least 140° C. and very preferentially at least 150° C.;
a gasoline cut whose upper (final) distillation point is preferably less than 140° C.

In this embodiment, the production of kerosene and gasoline is maximized.

According to another particular embodiment, the optional separation step e) advantageously makes it possible to obtain:
a top cut which corresponds to a gasoline advantageously comprising hydrocarbons containing between 5 and 10 carbon atoms;
an intermediate cut advantageously comprising hydrocarbons containing between 9 and 24 and preferably between 9 and 16 carbon atoms, and which constitutes a kerosene cut meeting the commercial specifications;
a “residue” cut, with an initial boiling point above 300° C., the desired final cut being kerosene, and which advantageously joins the diesel or fuel oil pool.

According to another particular embodiment, the optional separation step e) advantageously makes it possible to obtain:
a kerosene cut whose lower (initial) distillation point is preferably at least 140° C. and very preferentially at least 150° C., and whose final distillation point is less than or equal to 300° C., which constitutes a kerosene base meeting the commercial specifications;
optionally, a “residue” cut, with an initial boiling point above 300° C., which advantageously joins the diesel or fuel oil pool.

In this embodiment, the production of kerosene is maximized.

According to yet another particular embodiment, the optional separation step e) advantageously makes it possible to obtain:
a top cut which corresponds to a gasoline advantageously comprising hydrocarbons containing between 5 and 10 carbon atoms;
an intermediate cut advantageously comprising hydrocarbons containing between 10 and 24 carbon atoms, and which constitutes a gas oil cut meeting the commercial specifications.

In this embodiment, the production of gas oil is maximized.

Thus, the process according to the invention thus makes it possible to improve the selectivity of a light olefin oligomerization process towards middle distillates, in particular kerosene and/or gas oil, and thus to maximize the middle distillate yields, while at the same time having an optimum overall conversion of the olefinic feedstock. The process according to the invention is a particularly flexible process since a person skilled in the art can adapt the selectivity of the oligomerization and the separation of the effluents so as to maximize the production of kerosene and/or gas oil, up to the point of possibly producing only gas oil or only kerosene.

The examples and figures that follow illustrate the invention, notably particular embodiments of the invention, without limiting the scope thereof.

### LIST OF FIGURES

FIG. 1 represents diagrammatically an implementation of the process according to the invention.

A feedstock (1) rich in C3 and C4 olefins is treated in an oligomerization section (a). The reaction effluent 5 is sent to a separation step (b) and separated in a series of columns to produce:
a stream 4 rich in C3 to C4 compounds comprising the unconverted feedstock olefins and any feedstock constituents (e.g. paraffins) that do not react in this boiling range;
a stream 3 which comprises the dimers and trimers produced during oligomerization but which are too light to be upgraded as middle distillates;
a stream 9 recovered at the bottom of the last column and corresponding to the heavy fraction.

Stream 4 is at least partly recycled to the oligomerization inlet (a). Part of the stream (4) can be purged or upgraded in another unit (stream 6), either continuously or from time to time, depending on the nature of the feedstock.

Stream 3 is sent to the oligomerization step, at least in part, preferably totally, to the oligomerization inlet (a). Part of stream 3 may also be sent for upgrading to a gasoline pool (stream 8). Stream 8 may be optionally sent for hydrogenation c) and subsequently upgraded with stream 11.

Stream 9 is sent to a hydrogenation section (c). The hydrogenated effluent 10 is then separated in a section (d), into:
a stream 11 which is sent to a gasoline pool;
a stream 13 which is upgraded to kerosene;
a stream 14 consisting of the heaviest compounds produced during the process and which can be upgraded to gas oil.

An optional stream 2 consisting, for example, of a mixture of kerosene and gas oil (stream 12) is sent to the oligomerization step a). It corresponds to a stream of inerts used to control the exothermicity of the reaction in the reactors of the oligomerization section a).

### EXAMPLES

### Example 1 (in Accordance with the Invention)

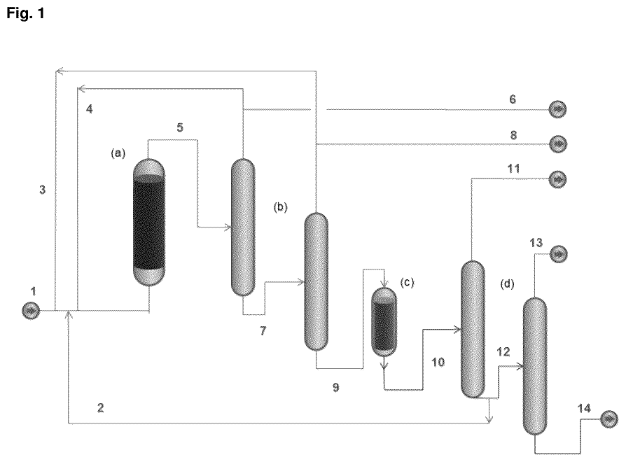

A C4 olefinic hydrocarbon feedstock, comprising 24.8% by weight of 1-butene, 75% by weight of 2-butenes and 0.2% by weight of n-butane, is oligomerized according to the process embodiment described in FIG. 1, in the presence of a silica-alumina catalyst (Axens commercial catalyst IP 811), at a temperature of between 14° and 190° C., a pressure of 3.5 MPa and an HSV of 0.3 h 1. The oligomerization reaction is performed in three reactors in series, with an intermediate heat exchanger between each reactor, allowing cooling before entry into the next reactor.

The reaction effluent obtained at the end of the oligomerization step is separated by distillation into three cuts:
1) a C4-cut, comprising the unreacted feedstock and corresponding to about 17% by weight of the reaction effluent, said C4 cut being entirely recycled to the oligomerization step (and corresponding to a weight ratio of the C4-cut relative to the fresh C4 olefinic hydrocarbon feedstock of between 0.8 and 1.0);
2) a C5-140° C. cut, constituting the intermediate fraction and corresponding to about 64% by weight of the reaction effluent, said C5-140° C. cut being entirely recycled to the inlet of the oligomerization step so that the weight ratio of the C5-140° C. cut relative to the fresh C4 olefinic hydrocarbon feedstock is equal to 3.6;
3) a 140-300° C. cut, corresponding to 19% by weight of the reaction effluent, which is sent for hydrogenation.

The conversion of the olefinic feedstock is greater than or equal to 90% by weight.

The hydrogenation is performed in the presence of a nickel catalyst on an alumina support, at 180° C. under 3.0 MPa of hydrogen with an HSV of 0.5 h 1 and a hydrogen flow rate of 50 NL/h.

The olefin content observed after hydrogenation is very low (bromine number <0.8 g/100 g), meaning that the degree of hydrogenation is high (greater than 99%).

The hydrogenation effluent is then sent to a distillation section where it is separated into three cuts:
a light gasoline cut with a top distillation point below 140° C. at a yield of 7% by weight relative to the weight of the olefins in the initial C4 olefinic hydrocarbon feedstock;
a kerosene cut, with a distillation range of 140° C.-300° C., in a yield of 86% by weight relative to the weight of the olefins of the C4 olefinic hydrocarbon feedstock; and
a 300+ residue corresponding to 7% by weight relative to the weight of the olefins of the C4 olefinic hydrocarbon feedstock.

### Example 2 (in Accordance with the Invention)

A biobased C4 olefinic hydrocarbon feedstock (obtained from the dehydration of isobutanol obtained by fermentation of sugars), comprising 94.5% by weight of isobutene and 5.5% by weight of isobutane is oligomerized in the presence of a silica-alumina catalyst (Axens commercial catalyst IP 811), at a temperature of between 3° and 90° C., a pressure of 3.5 MPa and an HSV of 0.3 h 1. The oligomerization reaction is performed in three reactors in series, with an intermediate heat exchanger between each reactor, allowing cooling before entry into the next reactor. Part of the hydrogenated end product is recycled to the oligomerization step a), so as to control the exothermicity in the reactors. This recycle represents 3.5 times the amount of fresh olefinic feedstock by weight.

The reaction effluent obtained at the end of the oligomerization step is separated by distillation into four cuts:
1) a C4-cut, comprising the unreacted feedstock and corresponding to about 7.1% by weight of the reaction effluent, said C4-cut being entirely recycled to the oligomerization step (corresponding to a weight ratio of the C4 cut to the fresh C4 olefinic hydrocarbon feedstock of between 0.4 and 0.6);
2) a C5-140° C. cut, constituting the intermediate fraction and corresponding to about 31.4% by weight of the reaction effluent, said C5-140° C. cut being entirely recycled to the inlet of the oligomerization step so that the weight ratio of the C5-140° C. cut to the fresh C4 olefinic hydrocarbon feedstock is equal to 2.0;
3) a 140-300° C. cut, corresponding to about 61.5% by weight of the reaction effluent, which is sent for hydrogenation.

The conversion of the olefinic feedstock is greater than or equal to 90% by weight.

The hydrogenation of the 140-300° C. cut is performed in the presence of a nickel catalyst on an alumina support, at 180° C. under 3.0 MPa of hydrogen with an HSV of 0.5 h¹ and a hydrogen flow rate of 50 NL/h.

The olefin content observed after hydrogenation is very low (bromine number <0.8 g/100 g), meaning that the degree of hydrogenation is high (greater than 99%).

The hydrogenation effluent is then sent to a distillation section where it is separated into three cuts:
a light gasoline cut with a top distillation point below 140° C. at a yield of 6% by weight relative to the weight of the olefins in the initial C4 olefinic hydrocarbon feedstock;
a kerosene cut, with a distillation range of 140° C.-300° C., in a yield of 89% by weight relative to the weight of the olefins of the C4 olefinic hydrocarbon feedstock; and
a 300+ residue corresponding to 5% by weight relative to the weight of the olefins of the C4 olefinic hydrocarbon feedstock.

### Example 3 (not in Accordance with the Invention)

A C4 olefinic hydrocarbon feedstock similar to that treated by means of the process described in Example 1 is treated in Example 3: it comprises 24.8% by weight of 1-butene, 75% by weight of 2-butenes and 0.2% by weight of n-butane.

The C4 olefinic hydrocarbon feedstock is oligomerized under operating conditions similar to those of the process described in Example 1. However, the C5-140° C. cut of the reaction effluent is not recycled to the inlet of the oligomerization step.

The reaction effluent obtained at the end of the oligomerization step is separated by distillation into three cuts:
1) a C4-cut, comprising the unreacted feedstock and corresponding to about 60.1% by weight of the reaction effluent, said C4 cut being partly returned to the oligomerization step such that the recycle ratio corresponds to a weight ratio of the C4-cut to the fresh C4 olefinic hydrocarbon feedstock equal to 0.5;
2) a C5-140° C. cut, constituting the intermediate fraction and corresponding to about 20.1% by weight of the reaction effluent;
3) a 140+ cut, corresponding to 19.8% by weight of the reaction effluent, which is sent for hydrogenation.

The conversion of the olefinic C4 compounds in the feedstock is about 85% by weight. The conversion of the olefinic feedstock to C4 (85% by weight) is lower than that obtained with the process described in Example 1 (at least 90% by weight).

The hydrogenation is performed under the same conditions as in Example 1.

The hydrogenation effluent is then sent to a distillation section where it is separated into three cuts:
a light gasoline cut with a top distillation point below 40° C. in a yield of 40% by weight relative to the olefins of the initial C4 olefinic hydrocarbon feedstock and;
a kerosene cut, with a distillation range of 140° C.-300° C., with a yield of 40% by weight relative to the olefins of the initial C4 olefinic hydrocarbon feedstock.

The yield of kerosene (40%) is lower than that obtained with the process described in Example 1 (86%).

1. Process for preparing middle distillates from an olefinic feedstock which comprises monoolefins containing between 3 and 6 carbon atoms, wherein the process comprises:
a) an oligomerization step which is fed with at least the olefinic feedstock, a first recycle and a second recycle, and which is operated in the presence of at least one oligomerization catalyst, at a temperature of between 2° and 500° C., a pressure of between 1.0 and 10 MPa and an HSV between 0.1 and 0.5 h⁻¹, to produce a reaction effluent comprising dimers, trimers and oligomers;
b) a step of fractionating the reaction effluent obtained at the end of step a), into at least:
a light fraction comprising at least part of the olefinic feedstock not converted in step a);
an intermediate fraction comprising at least part of the dimers and trimers produced in step a); and
a heavy fraction comprising the oligomers;
c) a recycle step, comprising: preparing a first recycle which comprises at least part of the light fraction; preparing a second recycle which comprises at least part of the intermediate fraction; and transferring the first recycle and the second recycle to the oligomerization step a);
d) a step for hydrogenating at least part of the heavy fraction separated out in step b) in the presence of hydrogen, to give a hydrogenated heavy fraction comprising middle distillates.

2. Process according to claim 1, in which the olefinic feedstock comprises monoolefins containing 3 and/or 4 carbon atoms.

3. Process according to claim 1, in which the olefinic feedstock is at least partly biobased.

4. Process according to claim 1, in which the first recycle feeds step a) in a weight ratio of between 0.3 and 1.5, preferably between 0.5 and 1.2, relative to the olefinic feedstock.

5. Process according to claim 1, in which the second recycle feeds step a) in a weight ratio of between 0.5 and 10.0, preferentially between 1.0 and 5.0 and preferably between 1.0 and 4.0, relative to the olefinic feedstock.

6. Process according to claim 1, in which the oligomerization catalyst in step a) is chosen from silica-impregnated phosphoric acid-based catalysts, ion-exchange resins, silica-alumina and pure zeolites or zeolites supported on alumina.

7. Process according to claim 1, in which the oligomerization step a) is performed in the presence of a silica-alumina-based catalyst and is performed at a temperature of between 20° C. and 300° C., preferentially between 25 and 220° C., preferably between 30° C. and 200° C.

8. Process according to claim 1, in which the oligomerization step a) is performed at a pressure of between 1.5 and 6.5 MPa, preferentially between 2.0 and 4.0 MPa.

9. Process according to claim 1, in which the oligomerization step a) is operated at an HSV of between 0.2 and 0.3 h⁻¹.

10. Process according to claim 1, also comprising a step e) of separating the hydrogenated heavy fraction obtained from step d), to separate out at least one middle distillate cut, in particular a kerosene cut and/or a gas oil cut.
